(19) United States
(12) Patent Application Publication
(10) Pub. No.: US 2009/0047328 A1
(43) Pub. Date: Feb. 19, 2009

(54) CAFFEINE DELIVERY SYSTEMS

(76) Inventor: Peter Cunningham, Las Vegas, NV (US)

Correspondence Address: GREENBERG TRAURIG, 3773 HOWARD HUGHES PARKWAY, SUITE 500 NORTH, LAS VEGAS, NV, 89169, US

(21) Appl. No.: 11/840,133

(22) Filed: Aug. 16, 2007

(51) Int. Cl. A61K 31/522; A61K 9/70

(52) U.S. Cl. 424/443; 514/263.31

(57) ABSTRACT

Described are caffeine-based delivery vehicles. A first delivery vehicle is a malleable strip that dissolves in an oral cavity. Buccal absorption provides a prompt mental and physical stimulation for the user. The strips are formulated with beneficial ingredients comprising caffeine, taurine and/or nitric oxide. The strips are contained in a small crush-proof package for convenience. A second delivery vehicle is a gel. The gel provides a prompt and sustained effect. The sustained effect based on gastric absorption of ingested gel that does not absorb through contact with a user's oral cavity. A high viscosity of the gel enhances the buccal absorption. Like the strips, the gel is formulated with beneficial ingredients comprising caffeine, taurine and/or nitric oxide. The gel is contained in packages having an integrated, re-sealable straw.

### FIELD OF THE INVENTION

The embodiments of the present invention relate to a caffeine strips and gel.

### BACKGROUND

Caffeine is a well-known stimulant found in coffee, power pills and energy drinks. In many instances, such items are ingested by users seeking an energy boost. However, each of the aforementioned delivery vehicles suffer from drawbacks.

Thus, there exists a need for caffeine delivery vehicles which overcome the drawbacks associated with currently available delivery vehicles.

### SUMMARY

Accordingly, a first embodiment of the present invention is a caffeine strip comprising a combination of: about 10% to 40% caffeine; about 0.5% to 5% sucralose; about 0.5% to 5% sodium citrate; about 5% to 15% taurine; about 0.5% to 5% soy lecithin; about 0.5% to 10% modified corn starch; about 0.5% to 10% sodium carboxymethyl cellulose; about 0.5% to 10% sorbitol; about 0.5% to 10% glycerol; about 5% to 40% sodium alginate; about 0.5% to 15% propylene glycol alginate; about 0.5% to 5% polysorbate 80; and about 0.5% to 15% flavor additive.

A second embodiment of the present invention is a caffeine gel comprising a combination of: about 0.05% to 5.0% caffeine; about 0.5% to 10% taurine; about 1% to 20% sugar; about 60% to 95% water; about 0.5% to 5% sodium citrate; about 0.5% to 5% enzyme modified sodium carboxymethyl cellulose; about 0.5% to 5% citric acid; about 0.5% to 5% guar gum; about 0.5% to 5% xanthan gum; about 0.5% to 5% gellan gum; and about 0.5% to 5% flavor additive.

The embodiments of the present invention provide more efficient vehicle delivery for caffeine such that less caffeine can be used to provide the same effect as other delivery vehicles using more caffeine. By using less of the key ingredients, the embodiments of the present invention allow the strips and gel to be fabricated less expensively than the other delivery vehicles.

Other variations, embodiments and features of the present invention will become evident from the following detailed description, drawings and claims.

### BRIEF DESCRIPTION OF THE DRAWINGS

FIG. 1 illustrates an ingredient list of caffeine/taurine strips according to one embodiment of the present invention;

FIG. 2 illustrates an exemplary nutritional label for a package of caffeine/taurine strips;

FIG. 3 illustrates an ingredient list of caffeine gel according to one embodiment of the present invention; and

FIG. 4 illustrates an exemplary nutritional label for a package of caffeine gel.

### DETAILED DESCRIPTION

It will be appreciated by those of ordinary skill in the art that the invention can be embodied in other specific forms without departing from the spirit or essential character thereof. The presently disclosed embodiments are therefore considered in all respects to be illustrative and not restrictive.

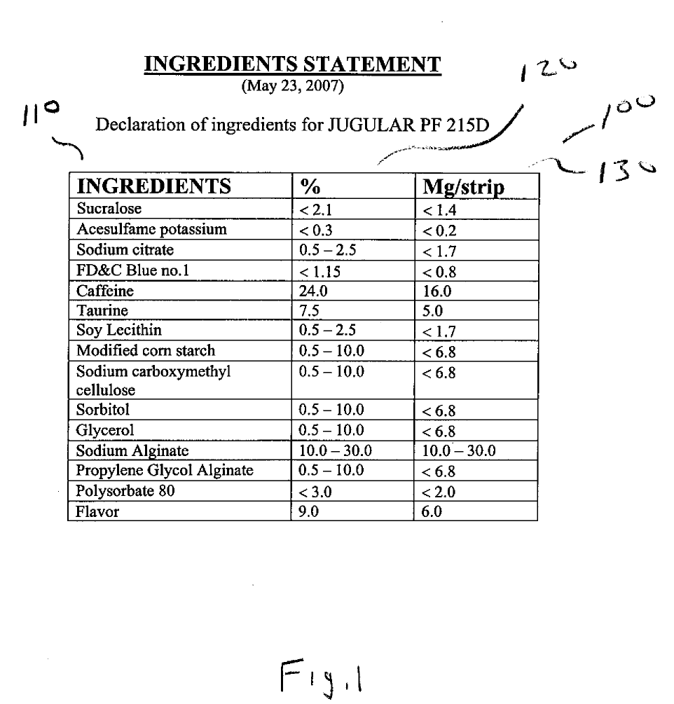

Initial reference is made to FIG. 1 illustrating an ingredient list 100 for a caffeine/taurine strip according to one embodiment of the present invention. The list 100 includes an ingredient column 110, % of ingredients column 120 and a weight of ingredients 130 per strip. As shown the list of ingredients includes sucralose, acesulfame potassium, sodium citrate, FD&C Blue No. 1, caffeine, taurine, soy lecithin, modified corn starch, sodium carboxymethyl cellulose, sorbitol, glycerol, sodium alginate, propylene glycol alginate, polysorbate 80 and a flavor additive. As shown, the strips have 16 mg of caffeine and 5 mg of taurine per strip. While the percentages of some ingredients are listed within a suitable range, it should be understood that all ingredients, including the active or beneficial ingredients have suitable ranges. Moreover, it will be understood by those skilled in the art that the ranges of ingredients and weights may be less than or greater than listed.

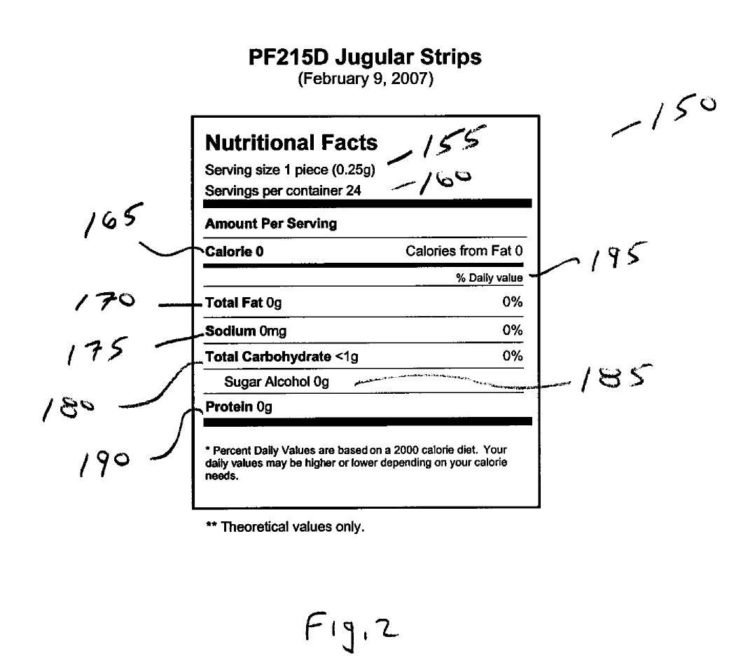

FIG. 2 shows an exemplary nutritional label 150 for a package containing twenty-four 0.25 g caffeine strips. The label 150 includes reference to a serving size 155, servings per container 160, calories per serving 165, fat per serving 170, sodium per serving 175, carbohydrates per serving 180, sugar alcohol per serving 185 and protein per serving 190. A listing of required % of daily value 195 is also set forth on the label 150. The label 150 may be modified in any manner desired by the manufacturer as long as it includes legally mandated information.

In an alternative embodiment, nitric oxide is an added ingredient used to formulate the caffeine strips. In one embodiment, 5 mg of nitric oxide is added to the strip formulation. It will be recognized by those skilled in the art that the amount of nitric oxide can be more or less than the 5 mg per strip. Nitric oxide improves vasodilatation (or vasorelaxation) thereby increasing blood flow and consequently nutrient supply to cells. The nitric oxide may increase alertness and may also combat the vasoconstrictive effects of other ingredients (e.g., caffeine) used in the strips. While nitric oxide is naturally occurring in the human body, the strips provide an enhanced amount beyond the amount made by the body.

The caffeine strips provide numerous benefits over other delivery vehicles including tablets and liquids. The strips are a food grade product that quickly dissolve in a users mouth. The low temperature and reduced moisture manufacturing process permits a significant loading of beneficial ingredients (e.g., caffeine and taurine) thereby increasing the benefit based on a weight by volume. The strips have a long shelf life in temperature ranging from 40 degrees Fahrenheit to 100 degrees Fahrenheit.

The malleability of the strips allows them to be folded and unfolded such that they retain their original shape. The malleability permits the user to suck, chew, melt or dissolve the strips in their oral cavity. The strips maintain their integrity upon being initially placed in the user's mouth. The strips then slowly dissolve in a user's mouth from pressure from a tongue pressing the strip against a user's cheek or the roof of the user's mouth or by chewing the strip. The strip dissolves immediately in the oral or buccal cavity at an optimal time for absorption of the active ingredients. As set forth below, there are many benefits associated with buccal absorption.

Buccal absorption provides a rapid and high rate of absorption into a users bloodstream providing a superior effect over gastric and/or liver absorption. As a result, user's feel a surge in mental and physical energy, from the caffeine, in approximately 30-60 seconds after consumption as compared to 30-40 minutes for ingested caffeine products. Advantageously, buccal absorption enables a relatively low weight by volume for the active ingredients to deliver the same effect as other delivery vehicles. Indeed, the low weight to volume strips provide enhanced effects over greater weight by volume configurations for ingested caffeine products. For example, a caffeine strip having 15 mg of caffeine has the same effect on the user as cup of coffee having 100 mg of caffeine. In one embodiment, the equivalent of 2 cups of coffee can be stored in crush-proof packaging measuring 2″×1″×¼ ″. The strips also contain no sugar, calories or carbohydrates unlike a cup of coffee which likely includes each. Also, the strips do not require the ingestion of liquids making them very convenient.

In one embodiment of the strips, flavoring is added. A combination of peppermint and vanilla is one example of suitable flavoring. Advantageously, peppermint/vanilla flavoring stimulates a high production of saliva to enhance the breakdown of the strip in the oral cavity while producing a fresh taste to effectively mask bitterness associated with caffeine. A myriad of other flavors are conceivable.

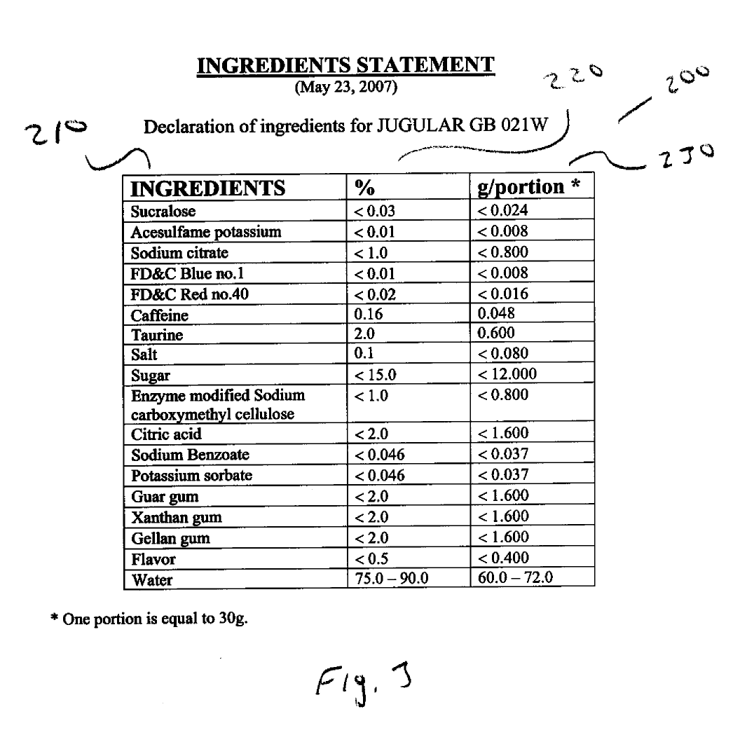

Reference is now made to FIG. 3 illustrating an ingredient list 200 for a caffeine/taurine gel according to one embodiment of the present invention. The list 200 includes an ingredient column 210, % of ingredients column 220 and a weight of ingredients 230 per gel package. As shown the list of ingredients includes sucralose, acesulfame potassium, sodium citrate, FD&C Blue No. 1, FD&C Red No. 40, caffeine, taurine, salt, sugar, enzyme modified sodium carboxymethyl cellulose, citric acid, sodium benzoate, potassium sorbate, guar gum xanthan gum, gellan gum, flavor and water. The percentages of each ingredient are listed within a suitable range. However, it will be understood by those skilled in the art that the ranges of ingredients and weights may be greater or less than listed.

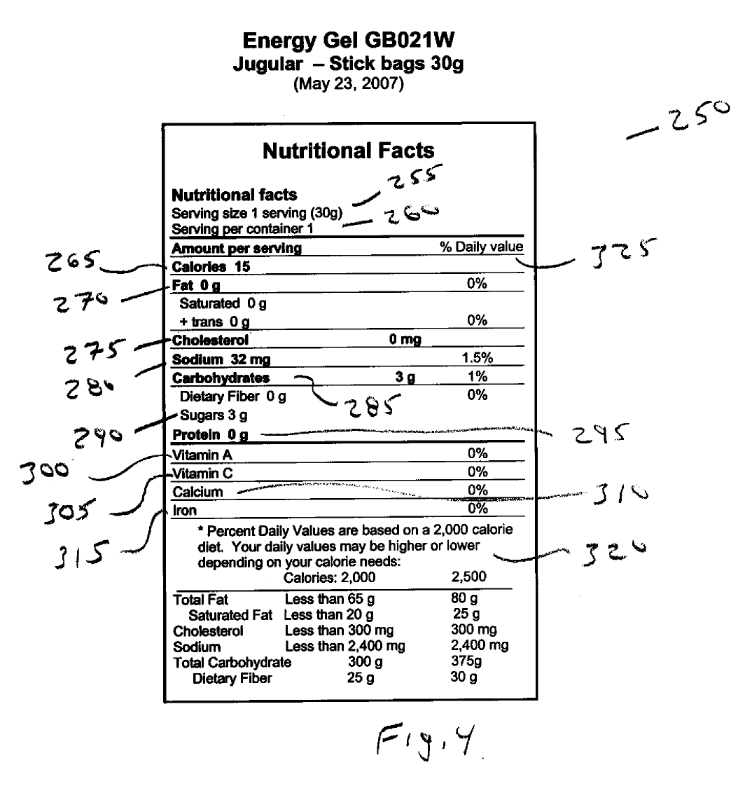

FIG. 4 shows an exemplary nutritional label 250 for a 30 g package of the gel. The gel may also be packaged in other sizes (e.g., 17 g and 80 g). In one embodiment, the gel packaging includes an integrated re-sealable straw to allow users to consume all or a portion of the gel directly from the package. The label 250 includes reference to a serving size 255, servings per container 260, calories per serving 265, fat per serving 270, cholesterol per serving 275, sodium per serving 280, carbohydrates per serving 285, sugars per serving 290, protein per serving 295, vitamin A per serving 300, vitamin C per serving 305, calcium per serving 310, iron per serving 315 and a breakdown 320 based on a caloric intake. A listing of required % of daily value 325 is also set forth on the label 250. The label 250 may be modified in any manner desired by the manufacturer as long as it includes legally mandated information.

The gel has generally the same benefits as the strips. The gel has a viscosity ranging from 2000 to 2500 centipoise (cps) permitting the gel to disperse throughpout an oral cavity maximizing its buccal contact and accelerating its absorption. Absorption typically occurs within 30 to 60 seconds. Secondary absorption occurs in the gastric tract within 20 to 40 minutes after ingestion. Therefore, the gel provides both short term and sustained effects. In one embodiment, the gel provides a 20% benefit within 30 seconds and a 100% benefit within 24 minutes.

In an alternative embodiment, nitric oxide is an added ingredient used to formulate the caffeine/taurine gel. Nitric oxide improves vasodilatation (or vasorelaxation) thereby increasing blood flow and consequently nutrient supply to cells. The nitric oxide may increase alertness and may also combat the vasoconstrictive effects of other ingredients (e.g., caffeine) used in the gel. While nitric oxide is naturally occurring in the human body, the strips provide an enhanced amount beyond the amount made by the body.

Although the invention has been described in detail with reference to several embodiments, additional variations and modifications exist within the scope and spirit of the invention as described and defined in the following claims.

I claim:

1. A caffeine strip comprising a combination of:
about 10% to 40% caffeine;
about 0.5% to 5% sucralose;
about 0.5% to 5% sodium citrate;
about 5% to 15% taurine;
about 0.5% to 5% soy lecithin;
about 0.5% to 10% modified corn starch;
about 0.5% to 10% sodium carboxymethyl cellulose;
about 0.5% to 10% sorbitol;
about 0.5% to 10% glycerol;
about 5% to 40% sodium alginate;
about 0.5% to 15% propylene glycol alginate;
about 0.5% to 5% polysorbate 80; and
about 0.5% to 15% flavor additive.

2. The caffeine strip of claim 1 further comprising about 5% to 25% nitric oxide.

3. The caffeine strip of claim 1 further comprising a peppermint/vanilla flavor additive.

4. The caffeine strip of claim 1 further comprising less than about 2% acesulfame potassium.

5. The caffeine strip of claim 1 further comprising less than about 3% FD&C Blue No. 1.

6. A caffeine strip comprising a combination of:
about 20% to 30% caffeine;
about 0.5% to 3% sucralose;
about 0.5% to 2.5% sodium citrate;
about 5% to 10% taurine;
about 0.5% to 2.5% soy lecithin;
about 0.5% to 10% modified corn starch;
about 0.5% to 10% sodium carboxymethyl cellulose;
about 0.5% to 10% sorbitol;
about 0.5% to 10% glycerol;
about 10% to 30% sodium alginate;
about 0.5% to 10% propylene glycol alginate;
about 0.5% to 5% polysorbate 80; and
about 5% to 12% flavor additive.

7. The caffeine strip of claim 6 further comprising about 5% to 25% nitric oxide.

8. The caffeine strip of claim 6 further comprising a peppermint/vanilla flavor additive.

9. The caffeine strip of claim 6 further comprising less than about 2% acesulfame potassium.

10. The caffeine strip of claim 6 further comprising less than about 3% FD&C Blue No. 1.

11. A caffeine gel comprising a combination of:
about 0.05% to 5.0% caffeine;
about 0.5% to 10% taurine;
about 1% to 20% sugar;
about 60% to 95% water;
about 0.5% to 5% sodium citrate;
about 0.5% to 5% enzyme modified sodium carboxymethyl cellulose;
about 0.5% to 5% citric acid;
about 0.5% to 5% guar gum;
about 0.5% to 5% xanthan gum;
about 0.5% to 5% gellan gum; and
about 0.5% to 5% flavor additive.

12. The caffeine gel of claim 11 further comprising one or more of the following:
about 0.01% to 0.05% sucralose;
about 0.01% FD&C Blue No. 1;
about 0.02% FD&C Red No. 40;
about 0.05% sodium benzoate; and
about 0.05% potassium sorbate.

13. The caffeine gel of claim 12 further comprising about 5% to 25% nitric oxide.

14. The caffeine gel of claim 12 further comprising a fruit punch flavor additive.

15. The caffeine gel of claim 12 wherein the gel viscosity ranges from about 1500 to 2750 centipoise.

16. A caffeine gel comprising a combination of:
about 0.05% to 1.0% caffeine;
about 0.5% to 5% taurine;
about 5% to 15% sugar;
about 75% to 90% water;
about 0.1% to 1% sodium citrate;
about 0.1% to 1% enzyme modified sodium carboxymethyl cellulose;
about 0.1% to 2% citric acid;
about 0.1% to 2% guar gum;
about 0.1% to 2% xanthan gum;
about 0.1% to 2% gellan gum; and
about 0.1% to 0.5% flavor additive.

17. The caffeine gel of claim 16 further comprising one or more of the following:
about 0.01% to 0.03% sucralose;
about 0.01% FD&C Blue No. 1;
about 0.02% FD&C Red No. 40;
about 0.05% sodium benzoate; and
about 0.05% potassium sorbate.

18. The caffeine gel of claim 17 further comprising about 5% to 10% nitric oxide.

19. The caffeine gel of claim 17 further comprising a fruit punch flavor additive.

20. The caffeine gel of claim 17 wherein the gel viscosity ranges from about 2000 to 2500 centipoise.
